(19) United States
(12) Patent Application Publication
(10) Pub. No.: US 2025/0027935 A1
(43) Pub. Date: Jan. 23, 2025

(54) MAGNETIC-FIELD BIOSENSOR MEASURING A DIFFERENTIAL SIGNAL FROM AT LEAST TWO MAGNETIC-FIELD SENSING ELEMENTS

(71) Applicant: Allegro MicroSystems, LLC, Manchester, NH (US)

(72) Inventors: Rémy Lassalle-Balier, Bures sur Yvette (FR); Alexander Latham, Harvard, MA (US); Paolo Campiglio, Arcueil (FR)

(73) Assignee: Allegro MicroSystems, LLC, Manchester, NH (US)

(21) Appl. No.: 18/355,536

(22) Filed: Jul. 20, 2023

(51) Int. Cl. G01N 33/543; B01L 3/00; G01N 27/74; G01R 33/12

(52) U.S. Cl. CPC G01N 33/54386; B01L 3/502761; G01N 27/745; G01N 33/54333; G01R 33/1269; B01L 2200/0652; B01L 2300/0883; B01L 2400/043

(57) ABSTRACT

In one aspect, a magnetic-field biosensor includes an insulator and a plurality of magnetic-field sensing elements that includes a first and a second magnetic-field sensing elements. The insulator has a first and a second plurality of portions, and the second plurality of portions is thicker than the first plurality of portions. The magnetic-field biosensor further includes a first receptor configured to attach to biological material and being on a first portion of the first plurality of portions and directly above the first magnetic-field sensing element; and a second receptor configured to attach to the biological material and being on a first portion of the second plurality of portions and directly above the second magnetic-field sensing element. Outputs of the first and the second magnetic-field sensing elements are used to sense a magnetic field from a first magnetic nanoparticle by reducing an effect of an applied magnetic field.

### BACKGROUND

Referring to FIG. 1, a magnetic-filed biosensor 100 includes transducers (e.g., a transducer 106a, a transducer 106b) on a substrate 102. The transducers 106a, 106b are encapsulated with an insulator 110 that prevents water absorption. A top surface of the insulator 110 includes receptors (e.g., a receptor 116), which can capture specific biological material (e.g., a biomaterial 118). In general, a fluid is poured on the surface of the insulator 110. If a specific biomaterial is present in the fluid, the specific biomaterial is captured by one or more of the receptors that are configured to attach to the specific biomaterial. The sensor 100 is later washed and a solution with magnetic nanoparticles (e.g., a magnetic nanoparticle 124a, magnetic nanoparticle 124b) that are configured to attach to the biomaterial 118 is poured on the sensor 100. If the biomaterial 118 is attached to one or more receptors, then the one or more of the magnetic nanoparticles are attached to each of the biomaterial and stay attached even after another wash of the magnetic biosensor 100.

Each of the magnetic nanoparticles generates a magnetic field. For example, the magnetic nanoparticle 124a generates a magnetic field 128a. The magnetic nanoparticles collectively behave like a super paramagnet and are configured to align with an externally applied magnetic field 120. Otherwise, the magnetization directions of the magnetic nanoparticles are randomly distributed. Each of the transducers 106a, 106b may be connected in series or in parallel to form a single device, and the transducers 106a, 106b are used to detect a corresponding magnetic field from corresponding magnetic nanoparticles equally and thereby detect the biomaterial 118. In this configuration, a magnetic field measured at the transducers is opposite to the externally applied magnetic field 120 (e.g., the magnetic field 128a measured at the transducer 106a is opposite to the applied magnetic field 120).

### SUMMARY

In one aspect, a magnetic-field biosensor includes a substrate and a plurality of magnetic-field sensing elements on the substrate. The plurality of magnetic-field sensing elements includes a first magnetic-field sensing element and a second magnetic-field sensing element. The magnetic-field biosensor also includes an insulator on the substrate and the plurality of magnetic-field sensing elements. The insulator has a first plurality of portions and a second plurality of portions, and the second plurality of portions is thicker than the first plurality of portions. The magnetic-field biosensor further includes a first receptor configured to attach to biological material and being on a first portion of the first plurality of portions and directly above the first magnetic-field sensing element; and a second receptor configured to attach to the biological material and being on a first portion of the second plurality of portions and directly above the second magnetic-field sensing element. The first magnetic-field sensing element is configured to detect a magnetic field from a first magnetic nanoparticle attached to the biological material that is attached to the first receptor. The second magnetic-field sensing element is configured to detect at least ten percent less of a magnetic field from a second magnetic nanoparticle attached to the biological material that is attached to the second receptor than the first magnetic-field sensing element detects from a first magnetic nanoparticle attached to the biological material that is attached to the first receptor. An output of the first magnetic-field sensing element and an output of the second magnetic-field sensing element are used to sense the magnetic field from the first magnetic nanoparticle by reducing an effect of an applied magnetic field.

In another aspect, a magnetic-field biosensor includes a substrate; and a plurality of magnetic-field sensing elements on the substrate. The plurality of magnetic-field sensing elements includes a first magnetic-field sensing element and a second magnetic-field sensing element. The magnetic-field biosensor also includes an insulator on the substrate and the plurality of magnetic-field sensing elements; a receptor configured to attach to biological material; and a deterrent layer on the insulator and configured to deter bonding of receptors. The receptor is attached to the insulator and directly above the first magnetic-field sensing element. The deterrent layer is directly above the second magnetic-field sensing element. The first magnetic-field sensing element is configured to detect a magnetic field from a magnetic nanoparticle attached to the biological material that is attached to the receptor, and an output of the first magnetic-field sensing element and an output of the second magnetic-field sensing element are used to sense the magnetic field from the magnetic nanoparticle by reducing an effect of an applied magnetic field.

In a further aspect, a magnetic-field biosensor includes a substrate forming a plurality of peaks comprising a first peak and a second peak, and forming a plurality of valleys, comprising a first valley, a second valley and a third valley. The magnetic-field biosensor also includes a plurality of magnetic-field sensing elements that include a first magnetic-field sensing element on a first peak of the substrate and a second magnetic-field sensing element on a second peak of the substrate. The magnetic-field biosensor further includes an insulator on the substrate and the plurality of magnetic-field sensing elements; and a plurality of deterrent layers configured to deter bonding of a biological material that include a first deterrent layer located on the first peak directly above the first magnetic-field sensing element, and a second deterrent layer located on the second peak directly above the second magnetic-field sensing element The first peak is between the first valley and the second valley, and the second peak is between the second valley and the third valley. The magnetic-field biosensor also includes a plurality of receptors configured to attach to biological material that include a first receptor attached to the insulator in the first valley, and a second receptor attached to the insulator in the second valley. The first magnetic-field sensing element receives a magnetic field, parallel to an applied magnetic field, from a first magnetic nanoparticle attached to the biological material that is attached to the first receptor. The second magnetic-field sensing element receives a magnetic field, parallel to the applied magnetic field, from a second magnetic nanoparticle, the second magnetic nanoparticle being attached to the biological material that is attached to the second receptor. An output of the first and the second magnetic-field sensing elements are used to sense the magnetic field from the first and the second magnetic nanoparticles by reducing an effect of an applied magnetic field.

### DESCRIPTION OF THE DRAWINGS

The foregoing features may be more fully understood from the following description of the drawings. The drawings aid in explaining and understanding the disclosed technology. Since it is often impractical or impossible to illustrate and describe every possible embodiment, the provided figures depict one or more illustrative embodiments. Accordingly, the figures are not intended to limit the scope of the broad concepts, systems and techniques described herein. Like numbers in the figures denote like elements.

FIG. 1 is a cross-sectional diagram of a prior art magnetic-field biosensor;

FIG. 2A is a cross-sectional diagram of an example of a novel magnetic-field biosensor;

FIG. 2B is a cross-sectional diagram of another example of the novel magnetic-field biosensor of FIG. 2A;

FIG. 3A is a cross-sectional diagram of another example of a novel magnetic-field biosensor;

FIG. 3B is a cross-sectional diagram of another example of the novel magnetic-field biosensor of FIG. 3A;

FIG. 4A is a cross-sectional diagram of a novel magnetic-field biosensor subcomponent;

FIG. 4B is a cross-sectional diagram of another example of the novel magnetic-field biosensor subcomponent of FIG. 4A;

FIG. 5A is a diagram of a further example of a novel magnetic-field biosensor;

FIG. 5B is a diagram of another example of the novel magnetic-field biosensor of FIG. 5A;

FIG. 6 is a diagram of an example of configurations of magnetic-field sensing elements in a matrix;

FIG. 7A is a circuit diagram of an example of a half bridge; and

FIG. 7B is a circuit diagram of an example of a full bridge.

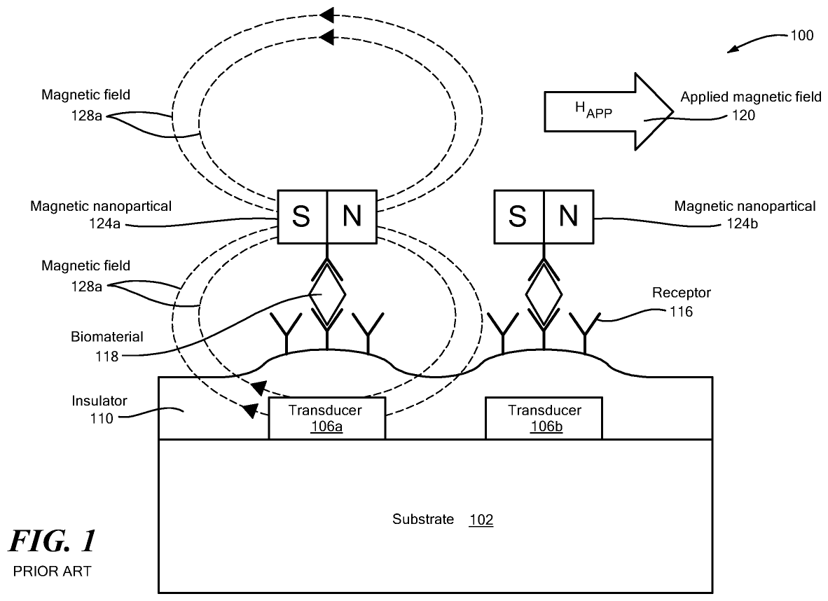

### DETAIL DESCRIPTION

Described herein are techniques to fabricate a magnetic-field biosensor that includes at least two magnetic-field sensing elements. In one example, a difference between an output of a first magnetic-field sensing element and an output of a second magnetic-field sensing element is used to detect a presence of at least one magnetic nanoparticle attached to a biomaterial. In one example, the applied magnetic-field, which is greater than the magnetic-field generated from a magnetic nanoparticle, is mitigated allowing for a better detection of the magnetic field of the magnetic nanoparticle. In one example, the difference between the electrical changes of the first magnetic-field sensing element and the electrical changes of the second magnetic-field sensing element is measured using a half bridge. In another example, the magnetic-field biosensor includes at least four magnetic-field sensing elements in a full bridge, and the presence of at least one magnetic nanoparticle attached to the biomaterial is detected using the differential output of the full bridge.

The biosensors described herein are subject to an AC magnetic field generated by a coil. Individual detection of a magnetic nanoparticle is nearly impossible as would be used in traditional methods. However, the biosensors described herein use a differential signal from a half or a full bridge that eliminates the effect of the AC magnetic field. Thus, the detection of magnetic nanoparticles is easier than the traditional methods.

As used herein, the term “magnetic-field sensing element” is used to describe a variety of electronic elements that can sense a magnetic field. The magnetic-field sensing element can be, but is not limited to, a Hall Effect element, a magnetoresistance element, a magnetotransistor or an inductive coil. As is known, there are several types of Hall Effect elements, for example, a planar Hall element, a vertical Hall element, and a Circular Vertical Hall (CVH) element.

As is also known, there are several types of magnetoresistance elements, for example, a semiconductor magnetoresistance element such as Indium Antimonide (InSb), a giant magnetoresistance (GMR) element, an anisotropic magnetoresistance element (AMR), a tunneling magnetoresistance (TMR) element, and a magnetic tunnel junction (MTJ).

The magnetic field sensing element may be a single element or, alternatively, may include two or more magnetic-field sensing elements arranged in various configurations, e.g., a half bridge or full (Wheatstone) bridge. Depending on the device type and other application requirements, the magnetic-field sensing element may be a device made of a type IV semiconductor material such as Silicon (Si) or Germanium (Ge), or a type III-V semiconductor material like Gallium-Arsenide (GaAs) or an Indium compound, e.g., Indium-Antimonide (InSb).

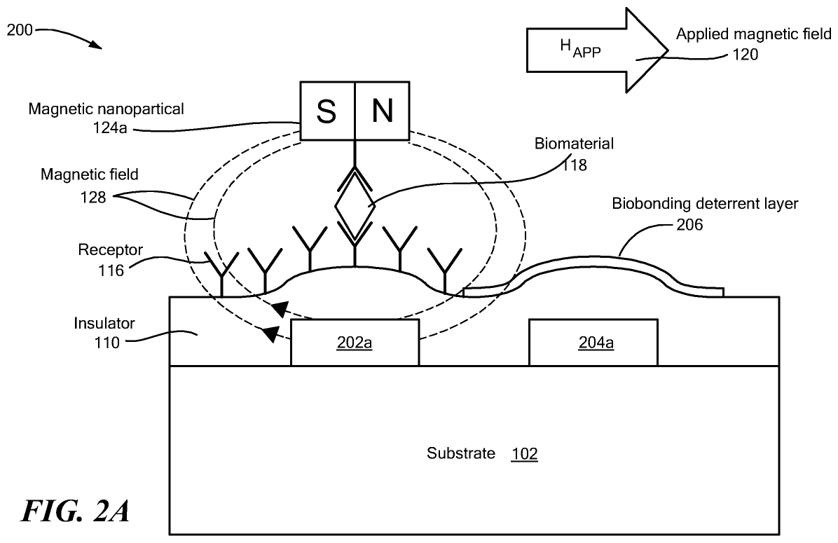

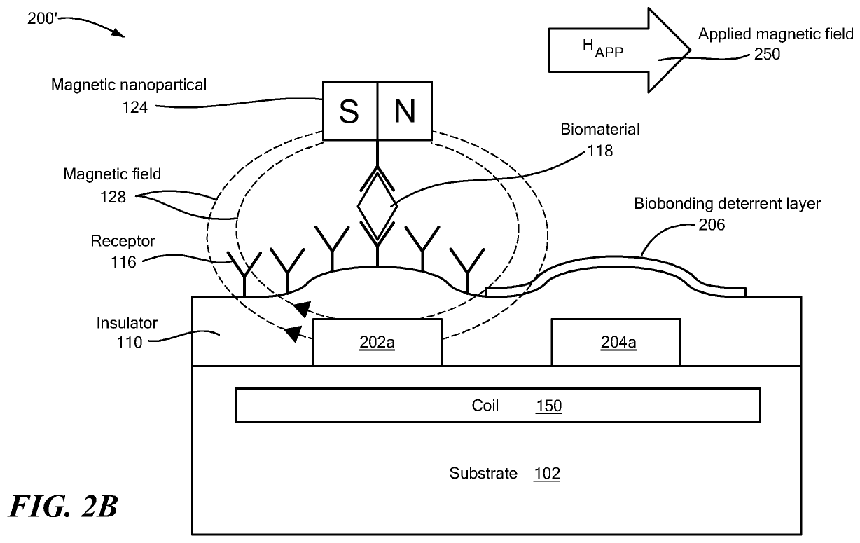

Referring to FIG. 2A, a magnetic-field biosensor 200 includes the substrate 102 with at least two magnetic-field sensing elements (e.g., a magnetic-field sensing element 202a, a magnetic-field sensing element 204a) on a top surface of the substrate 102. The at least two magnetic-field sensing elements are encapsulated in the insulator 110. Receptors (e.g., the receptor 116) are attached to the top surface of the insulator 110 directly above the magnetoresistance element 202a. A biobonding deterrent layer 206 is disposed on the top surface of the insulator 110 directly above the magnetic-field sensing element 204a. The biobonding deterrent layer 206 prevents any receptors from attaching to the biobonding deterrent layer 206. In one example, the biobonding deterrent layer 206 is a layer of octadecyltrichlorosilane.

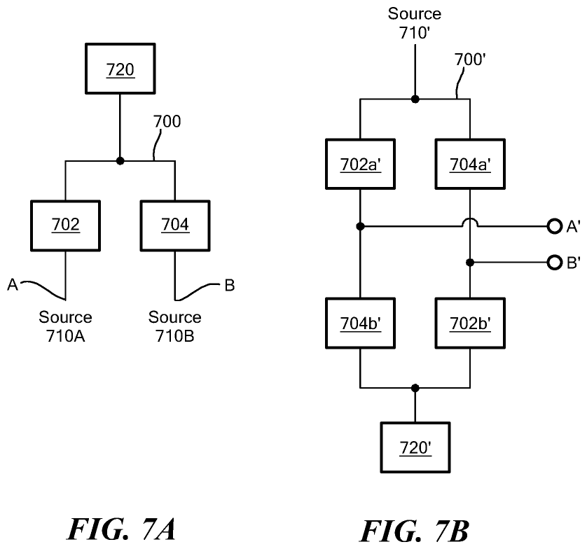

In this configuration, the magnetic-field sensing element 202a detects more of the magnetic field 128 from the magnetic nanoparticle 124 than the magnetic-field sensing element 204a detects the magnetic field 128 from the magnetic nanoparticle 124. In one example, a detection of the magnetic field 128 of the magnetic nanoparticle 124 (and hence, the detection of the biomaterial 118) is performed by taking a difference of electrical changes of the magnetic-field sensing element 202a and electrical changes of the magnetic-field sensing element 204a by placing the magnetic-field sensing elements 202a, 204a in a half bridge (see, for example, a half-bridge 700 in FIG. 7A).

In other examples, the magnetic-field biosensor 200 in FIG. 2A may be expanded. In one example, the magnetic-field biosensor 200 may further include two more magnetic-field sensing elements where one of the additional magnetic-field sensing elements is located under another biobonding deterrent layer and the other one of the additional magnetic-field sensing elements is directly under additional one or more receptors. The additional components may extend into the page of FIG. 2A or be side-by-side with the components in FIG. 2A.

The four magnetic-field sensing elements may be disposed in a full bridge (see, for example, a full bridge 700′ in FIG. 7B). A differential output of the full bridge may be used to determine if magnetic nanoparticles exist.

In other examples, additional pairs of magnetic-field sensing elements may be further expanded into the page of FIG. 2A and/or side-by-side with the components in FIG. 2A where one of the additional magnetic-field sensing elements in a pair is located under a biobonding deterrent layer and the other one of the additional magnetic-field sensing elements in the pair is directly under one or more receptors. The magnetic-field sensing elements may be disposed in a full bridge (see, for example, the full bridge 700′ in FIG. 7B). A differential output of the full bridge may be used to detect if magnetic nanoparticles exist.

Referring to FIG. 2B, another example of the magnetic-field biosensor 200 is a magnetic-field biosensor 200′. The magnetic-field biosensor 200′ is the same as the magnetic-field biosensor 200 except the magnetic-field biosensor 200′ includes one or more coils 150 with a current used to generate an applied magnetic field 250.

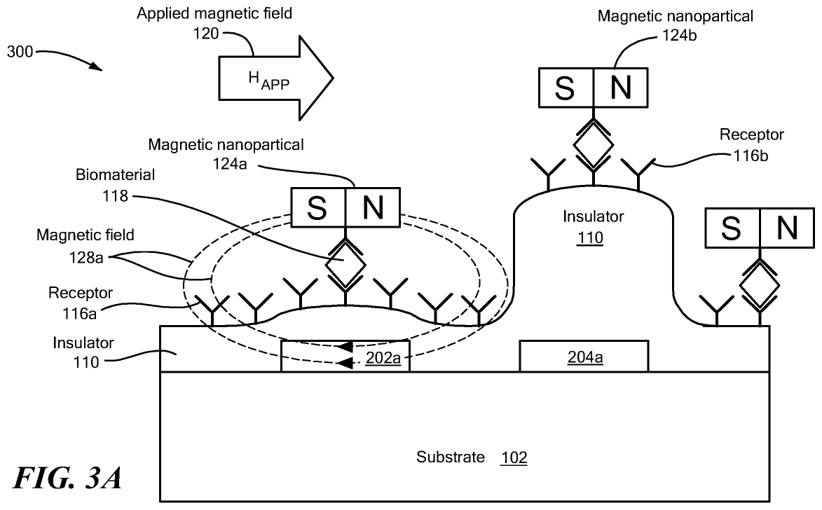

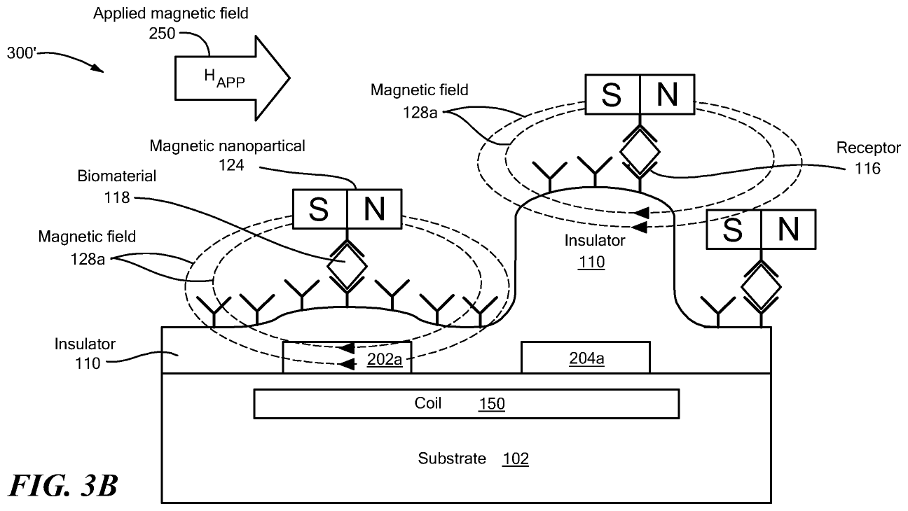

Referring to FIG. 3A, a magnetic-field biosensor 300 includes the same elements as the magnetic-field biosensor 200 except the magnetic-field biosensor 300 does not include the biobonding deterrent layer 206 above the magnetic-field sensing element 204a so that receptors may be attached directly above the magnetic-field sensing element 204a. The magnetic-field biosensor 300 includes a receptor 116a directly above the magnetic-field sensing element 202a and a receptor 116b directly above the magnetic-field sensing element 202b. Also, a thickness of the insulator 110 directly above the magnetic-field sensing element 204a is substantially thicker than a thickness of the insulator 110 directly above the magnetic-field sensing element 202a. Thus, the magnetic-field sensing element 204a is less sensitive than the magnetic-field sensing element 202a to the magnetic nanoparticles. In one particular example, the second magnetic-field sensing element 204a is configured to detect at least ten percent less of a magnetic field 128b from a second magnetic nanoparticle 124a indirectly attached to the receptor 116b than the first magnetic field sensing element 202a detects a magnetic field 128a from a first magnetic nanoparticle 124a indirectly attached to the receptor 116a.

A detection of the biomaterial 118 is performed by taking a difference of electrical changes of the magnetic-field sensing element 202a and electrical changes of the magnetic-field sensing element 204a by placing the magnetic-field sensing elements 202a, 204a in a half bridge (see, for example, the half bridge 700 in FIG. 7A).

In other examples, the magnetic-field biosensor 300 in FIG. 3A may be expanded. In one example, the magnetic-field biosensor 300 may further include two more magnetic-field sensing elements where one of the additional magnetic-field sensing elements is located under another biobonding deterrent layer and the other one of the additional magnetic-field sensing elements is directly under additional one or more receptors. The additional components may extend into the page of FIG. 3A or be side-by-side with the components in FIG. 3A.

The four magnetic-field sensing elements may be disposed in a full bridge (see, for example, the full bridge 700′ in FIG. 7B). A differential output of the full bridge may be used to determine if magnetic nanoparticles exist.

In other examples, additional pairs of magnetic-field sensing elements may be further expanded into the page of FIG. 3A and/or side-by-side with the components in FIG. 3A where one of the additional magnetic-field sensing elements in a pair is located under a biobonding deterrent layer and the other one of the additional magnetic-field sensing elements in the pair is directly under one or more receptors. The magnetic-field sensing elements may be disposed in a full bridge (see, for example, the full bridge 700′ in FIG. 7B). A differential output of the full bridge may be used to determine if magnetic nanoparticles exist.

Referring to FIG. 3B, another example of the magnetic-field biosensor 300 is a magnetic-field biosensor 300′. The magnetic-field biosensor 300′ is the same as the magnetic-field biosensor 300 except the magnetic-field biosensor 300′ includes one or more coils 150 with a current used to generate the applied magnetic field 250.

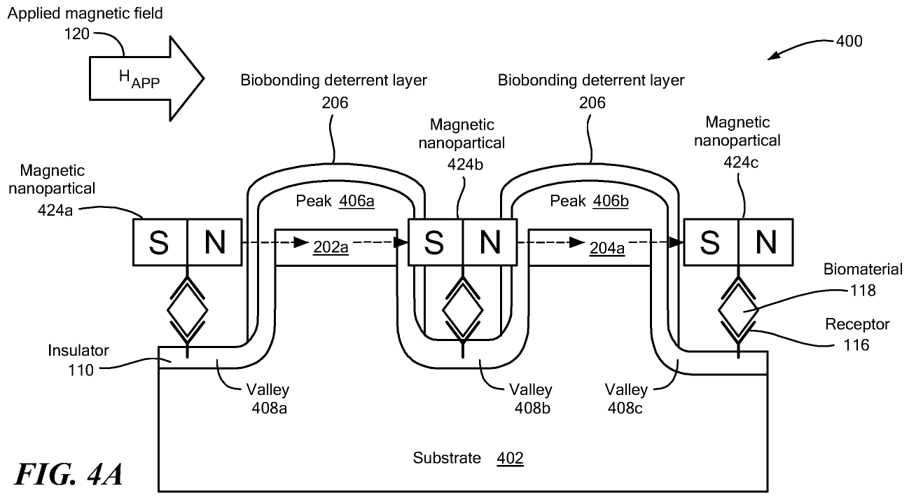

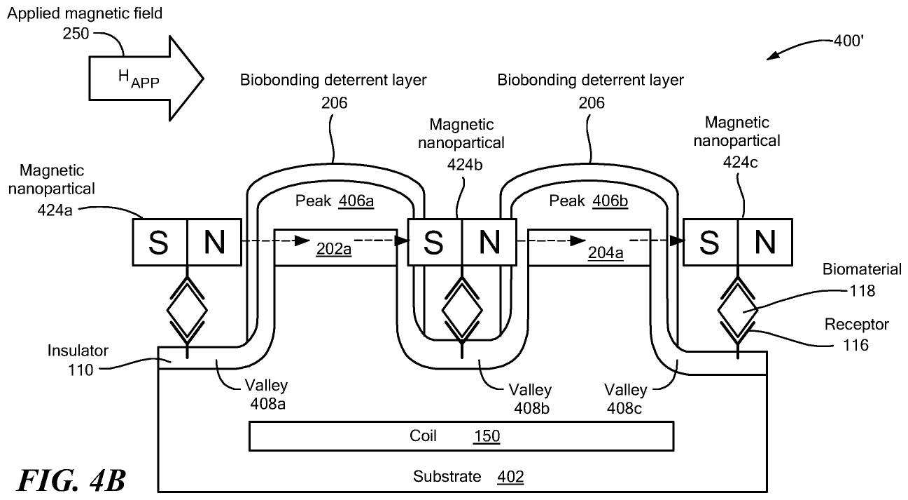

Referring to FIG. 4A, a magnetic-field biosensor subcomponent 400 includes a substrate 402 and the insulator 110 that together form peaks (a peak 406a, a peak 406b) and valleys (a valley 408a, a valley 408b, a valley 408c). The biobonding deterrent layer 206 is deposited on the peaks 406a, 406b but not in the valleys 408a-408c so that the receptors 116 are attached to the insulator 110 in the valleys 408a-408c.

In this configuration the magnetic nanoparticles (e.g., a magnetic nanoparticle 424a, a magnetic nanoparticle 424b, a magnetic nanoparticle 424c) are in a line with the magnetic-field sensing elements 202a, 204b forming a magnetic flux concentrator. The magnetic nanoparticles 424a, 424b provide a magnetic field 128 to the magnetic-field sensing elements 202a, 204a that are in the same direction (parallel) as the applied magnetic field 120.

In other examples, the magnetic-field biosensor subcomponent 400 in FIG. 4A may be expanded. In one example, the magnetic-field biosensor 400 may further include two more magnetic-field sensing elements where each of the additional magnetic-field sensing elements is located under another biobonding deterrent layer in a corresponding additional peak. The additional components may extend into the page of FIG. 4A or be side-by-side with the components in FIG. 4A.

The four magnetic-field sensing elements may be disposed in a full bridge (see, for example, the full bridge 700′ in FIG. 7B). A differential output of the full bridge may be used to determine if magnetic nanoparticles exist.

In other examples, additional pairs of magnetic-field sensing elements may be further expanded into the page of FIG. 4A and/or side-by-side with the components in FIG. 4A where each of the additional magnetic-field sensing elements is located under a biobonding deterrent layer in a corresponding additional peak.

The additional pairs of magnetic-field sensing elements may be disposed in a full bridge (see, for example, the full bridge 700′ in FIG. 7B). A differential output of the full bridge may be used to determine if magnetic nanoparticles exist.

Referring to FIG. 4B, another example of the magnetic-field biosensor subcomponent 400 is a magnetic-field biosensor subcomponent 400′. The magnetic-field biosensor subcomponent 400′ is the same as the magnetic-field biosensor subcomponent 400 except the magnetic-field biosensor subcomponent 400′ includes one or more coils 150 with a current used to generate the applied magnetic field 250.

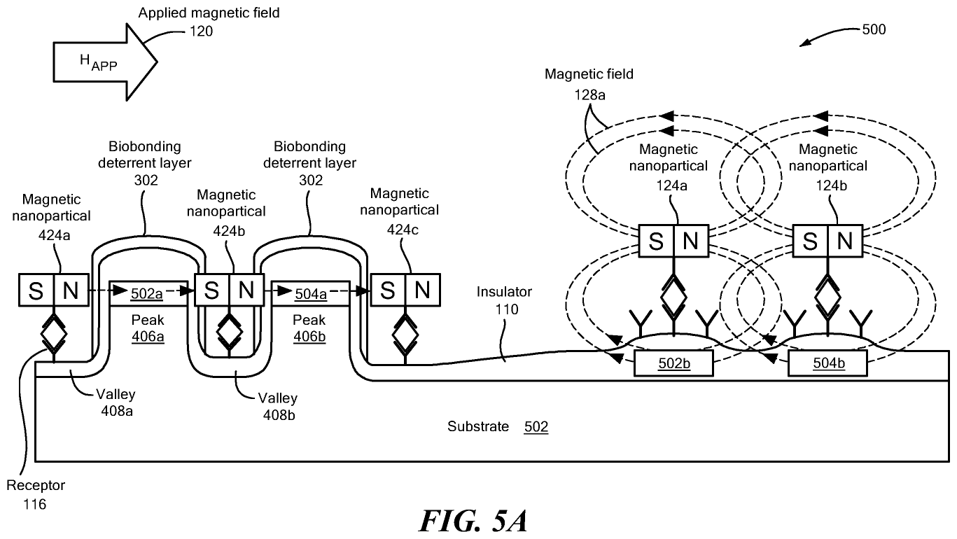

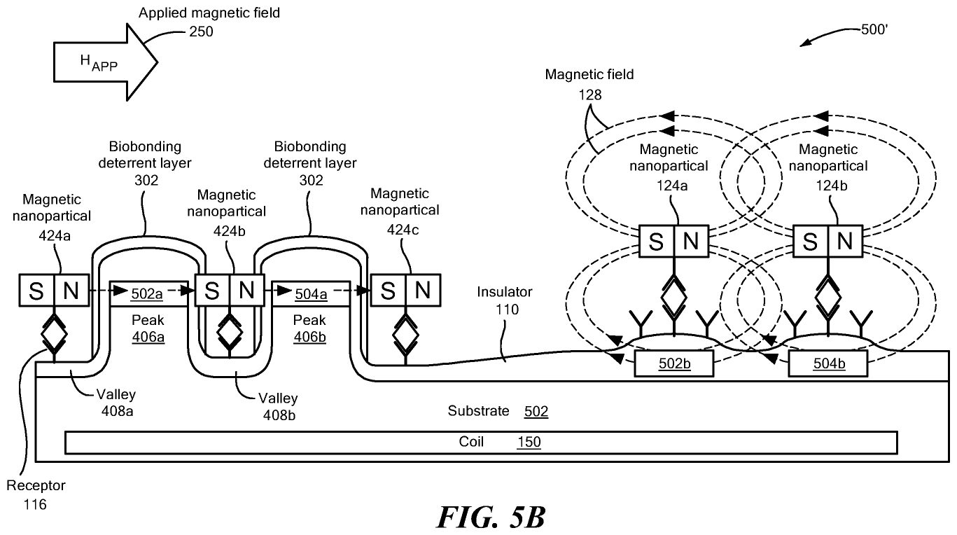

Referring to FIG. 5A, different combinations of the previous magnetic-field biosensors (i.e., magnetic-field biosensors 100, 200, 200′, 300, 300′) may be combined with the magnetic-field biosensor subcomponents 400, 400′to form other magnetic-field biosensors. In one particular example, the magnetic-field biosensor subcomponent 400 may be combined with the magnetic-field biosensor 100 to form a magnetic-field biosensor 500. The magnetic-field biosensor 500 includes at least four magnetic-field sensing elements (e.g., a magnetic-field sensing element 502a, a magnetic-field sensing element 502b, a magnetic-field sensing element 504a, a magnetic-field sensing element 504b).

In this configuration the magnetic nanoparticles (e.g., a magnetic nanoparticle 424a, a magnetic nanoparticle 424b, a magnetic nanoparticle 424c) are in a line with the magnetic-field sensing elements 502a, 504b forming a magnetic flux concentrator. The magnetic nanoparticles 424a-424b provide a magnetic field to the magnetic-field sensing elements 502a, 504a that are in the same direction (parallel) as the applied magnetic field 120. On the other hand, magnetic nanoparticles 124a, 124b provide a magnetic field 128a, 128b respectively to the magnetic-field sensing elements 502b, 504b that is opposite (antiparallel) to the applied magnetic field 120 and thereby mitigates the effects of the applied magnetic field 120.

The four magnetic-field sensing elements 502a, 502b, 504a, 504b may be disposed in a full bridge (see, for example, the full bridge 700′ in FIG. 7B). A differential output of the full bridge may be used to determine if magnetic nanoparticles exist.

Referring to FIG. 5B, another example of the magnetic-field biosensor 500 is a magnetic-field biosensor 500′. The magnetic-field biosensor 500′ is the same as the magnetic-field biosensor 500 except the magnetic-field biosensor 500′ includes one or more coils 150 with a current used to generate the applied magnetic field 250.

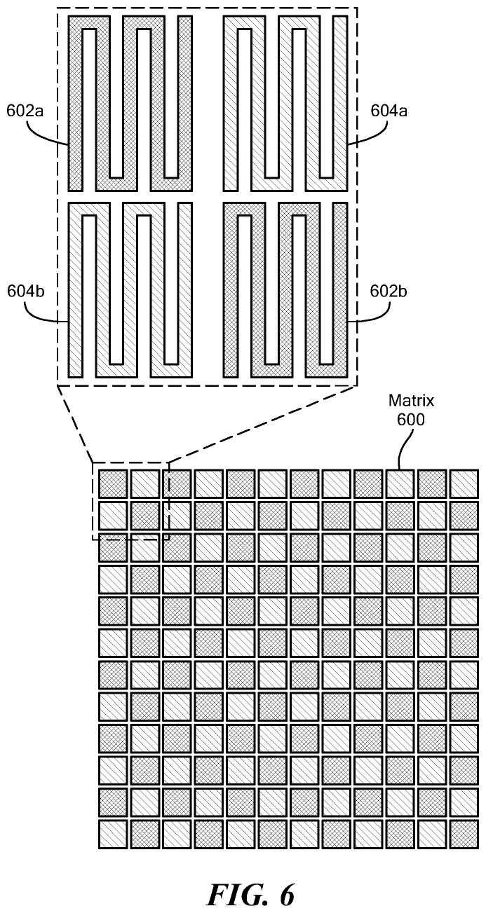

Referring to FIG. 6, a magnetic-field biosensor may be constructed using the techniques described herein in a matrix 600. Each pixel of the matrix 600 may include four magnetic-field sensing elements. In this particular example, each pixel includes four GMR elements (e.g., GMR element 602a, GMR element 602b, GMR element 604a, GMR element 604b).

In one example, each pixel may have a different type of receptor from the other pixels to detect a different biomaterial. In one particular example, a magnetic-field biosensor may be constructed to detect many types of allergens or cancers at a time.

Referring to FIG. 7A, the magnetic-field sensing elements may be connected in a half bridge. For example, a half bridge 700 includes a magnetic-field sensing element 702 on one leg of the half bridge 700 and a magnetic-field sensing element 704 on other leg of the half bridge 700. The half bridge 700 includes a source 710a at a node A directly connected to the magnetic-field sensing element 702, and a source 710b at a node B directly connected to the magnetic-field sensing element 704. The magnetic-field sensing elements 702, 704 are connected to ground 720.

In one example, the sources 710a. 710b are current sources. The voltage difference is measured between node A and node B. In another example, the sources 710a, 710b are voltage sources. The current difference is measured between node A and node B.

Referring to FIG. 7B, the magnetic-field sensing elements may be connected in a full bridge. For example, a full bridge 700′ includes a magnetic-field sensing element 702a and magnetic-field sensing element 704b on one leg of the bridge 700′, and a magnetic-field sensing element 704a′ and magnetic-field sensing element 702b′ on the other leg of the full bridge 700′. The bridge 700′ includes a source 710′ directly connected to the magnetic-field sensing elements 702a′, 702b′, and a ground 720′ directly connected to the magnetic-field sensing elements 704a′, 704b′. In one example, the source 710′ is a current source. In another example, the source 710′ is a voltage source.

A node A′ is between the magnetic-field sensing elements 702a, 704b, and a node B′ is between the magnetic-field sensing elements 704a, 702b. The difference between the node A′ and the node B′ is a differential output of the bridge 700′.

The embodiments are not limited to the embodiments described herein. For example, one or more coils may be used. Also, coils may be used in a substrate or external to the substrate.

Having described preferred embodiments, which serve to illustrate various concepts, structures, and techniques, which are the subject of this patent, it will now become apparent to those of ordinary skill in the art that other embodiments incorporating these concepts, structures and techniques may be used.

Elements of different embodiments described herein may be combined to form other embodiments not specifically set forth above. Various elements, which are described in the context of a single embodiment, may also be provided separately or in any suitable subcombination. Other embodiments not specifically described herein are also within the scope of the following claims.

What is claimed is:

1. A magnetic-field biosensor comprising:
a substrate;
a plurality of magnetic-field sensing elements on the substrate, the plurality of magnetic-field sensing elements comprising:
a first magnetic-field sensing element; and
a second magnetic-field sensing element;
an insulator on the substrate and the plurality of magnetic-field sensing elements, the insulator having a first plurality of portions and a second plurality of portions, wherein the second plurality of portions is thicker than the first plurality of portions;
a first receptor configured to attach to biological material, the first receptor being on a first portion of the first plurality of portions and directly above the first magnetic-field sensing element; and
a second receptor configured to attach to the biological material, the second receptor being on a first portion of the second plurality of portions and directly above the second magnetic-field sensing element, wherein the first magnetic-field sensing element is configured to detect a magnetic field from a first magnetic nanoparticle attached to the biological material that is attached to the first receptor,
wherein the second magnetic-field sensing element is configured to detect at least ten percent less of a magnetic field from a second magnetic nanoparticle attached to the biological material that is attached to the second receptor than the first magnetic-field sensing element detects from a first magnetic nanoparticle attached to the biological material that is attached to the first receptor, and
wherein an output of the first magnetic-field sensing element and an output of the second magnetic-field sensing element are used to sense the magnetic field from the first magnetic nanoparticle by reducing an effect of an applied magnetic field.

2. The magnetic-field biosensor of claim 1, further comprising at least one coil in the substrate or external to the substrate,
wherein the applied magnetic field is generated by the at least one coil.

3. The magnetic-field biosensor of claim 1, wherein the plurality of magnetic-field sensing elements further comprises:
a third magnetic-field sensing element; and
a fourth magnetic-field sensing element, further comprising:
a third receptor configured to attach to the biological material, the third receptor being on a second portion of the first plurality of portions and directly above the third magnetic-field sensing element; and
a fourth receptor configured to attach to the biological material, the fourth receptor being on a second portion of the second plurality of portions and directly above the fourth magnetic-field sensing element,
wherein the third magnetic-field sensing element is configured to detect a magnetic field from a third magnetic nanoparticle attached to the biological material that is attached to the third receptor, and
wherein the fourth magnetic-field sensing element is configured to detect at least ten percent less of a magnetic field from a fourth magnetic nanoparticle attached to the biological material that is attached to the fourth receptor than the third magnetic-field sensing element detects from a third magnetic nanoparticle attached to the biological material that is attached to the third receptor.

4. The magnetic-field biosensor of claim 3, wherein the first, the second, the third and the fourth magnetic-field sensing elements are in a full bridge.

5. The magnetic-field biosensor of claim 1, wherein the first and second magnetic-field sensing elements are in a half bridge.

6. The magnetic-field biosensor of claim 1, wherein the first and second magnetic-field sensing elements are in a full bridge.

7. The magnetic-field biosensor of claim 1, wherein the first and the second magnetic-field sensing elements are each a giant magnetoresistance element (GMR).

8. The magnetic-field biosensor of claim 1, wherein the first and the second magnetic-field sensing elements are each serpentine-shaped.

9. The magnetic-field biosensor of claim 1, wherein the first and the second magnetic-field sensing elements are each a tunneling magnetoresistance (TMR) element.

10. The magnetic-field biosensor of claim 1, wherein the biological material is a piece of deoxyribonucleic acid (DNA), a piece of ribonucleic acid (RNA), a protein, a virus, a biological cell, an antibody and/or a biological molecule.

11. A magnetic-field biosensor comprising:
a substrate;
a plurality of magnetic-field sensing elements on the substrate, the plurality of magnetic-field sensing elements comprising:
a first magnetic-field sensing element; and
a second magnetic-field sensing element;
an insulator on the substrate and the plurality of magnetic-field sensing elements;
a receptor configured to attach to biological material, the receptor being attached to the insulator and directly above the first magnetic-field sensing element; and
a deterrent layer on the insulator and configured to deter bonding of receptors, the deterrent layer being directly above the second magnetic-field sensing element,
wherein the first magnetic-field sensing element is configured to detect a magnetic field from a magnetic nanoparticle attached to the biological material that is attached to the receptor, and
wherein an output of the first magnetic-field sensing element and an output of the second magnetic-field sensing element are used to sense the magnetic field from the magnetic nanoparticle by reducing an effect of an applied magnetic field.

12. The magnetic-field biosensor of claim 11, further comprising at least one coil in the substrate or external to the substrate,
wherein the applied magnetic field is generated by the at least one coil.

13. The magnetic-field biosensor of claim 11, wherein the receptor is a first receptor, the magnetic nanoparticle is a first magnetic nanoparticle,
further comprising:
a third magnetic-field sensing element;
a fourth magnetic-field sensing element; and
a second receptor configured to attach to the biological material, the second receptor being on the insulator and directly above the third magnetic-field sensing element,
wherein the third magnetic-field sensing element is configured to detect a magnetic field from a second magnetic nanoparticle attached to the biological material that is attached to the second receptor.

14. The magnetic-field biosensor of claim 13, wherein the deterrent layer is a first deterrent layer,
further comprising a second deterrent layer that deters bonding of receptors, the second deterrent layer being directly above the fourth magnetic-field sensing element.

15. The magnetic-field biosensor of claim 13, wherein the first, the second, the third and the fourth magnetic-field sensing elements are in a full bridge.

16. The magnetic-field biosensor of claim 11, wherein the first and second magnetic-field sensing elements are in a half bridge.

17. The magnetic-field biosensor of claim 11, wherein the first and second magnetic-field sensing elements are in a full bridge.

18. The magnetic-field biosensor of claim 11, wherein the first and the second magnetic-field sensing elements are each a giant magnetoresistance element (GMR).

19. The magnetic-field biosensor of claim 11, wherein the first and the second magnetic-field sensing elements are each serpentine-shaped.

20. The magnetic-field biosensor of claim 11, wherein the first and the second magnetic-field sensing elements are each a tunneling magnetoresistance (TMR) element.

21. The magnetic-field biosensor of claim 11, wherein the biological material is a piece of deoxyribonucleic acid (DNA), a piece of ribonucleic acid (RNA), a protein, a virus, a biological cell, an antibody and/or a biological molecule.

22. The magnetic-field biosensor of claim 11, wherein the deterrent layer is a layer of octadecyltrichlorosilane.

23. A magnetic-field biosensor comprising:
a substrate forming a plurality of peaks comprising a first peak and a second peak, and forming a plurality of valleys, comprising a first valley, a second valley and a third valley;
a plurality of magnetic-field sensing elements comprising:
a first magnetic-field sensing element on a first peak of the substrate; and
a second magnetic-field sensing element on a second peak of the substrate;
an insulator on the substrate and the plurality of magnetic-field sensing elements;
a plurality of deterrent layers configured to deter bonding of a biological material, the plurality of deterrent layers comprising:
a first deterrent layer located on the first peak directly above the first magnetic-field sensing element, wherein the first peak is between the first valley and the second valley; and
a second deterrent layer located on the second peak directly above the second magnetic-field sensing element, wherein the second peak is between the second valley and the third valley; and
a plurality of receptors configured to attach to biological material, the plurality of receptor comprising:
a first receptor attached to the insulator in the first valley; and
a second receptor attached to the insulator in the second valley; and
wherein the first magnetic-field sensing element receives a magnetic field, parallel to an applied magnetic field, from a first magnetic nanoparticle attached to the biological material that is attached to the first receptor,
wherein the second magnetic-field sensing element receives a magnetic field, parallel to the applied magnetic field, from a second magnetic nanoparticle, the second magnetic nanoparticle being attached to the biological material that is attached to the second receptor,
wherein an output of the first magnetic-field sensing element and an output of the second magnetic-field sensing elements are used to sense the magnetic field from the first and the second magnetic nanoparticles by reducing an effect of an applied magnetic field.

24. The magnetic-field biosensor of claim 23, further comprising at least one coil in the substrate or external to the substrate,
wherein the applied magnetic field is generated by the at least one coil.

25. The magnetic-field biosensor of claim 23, wherein the plurality of magnetic-field sensing elements further comprises:
a third magnetic-field sensing element;
a fourth magnetic-field sensing element;
a third receptor configured to attach to the biological material, the third receptor being directly above the third magnetic-field sensing element; and
a fourth receptor configured to attach to the biological material, the fourth receptor being directly above the fourth magnetic-field sensing element,
wherein the third magnetic-field sensing element receives a magnetic field, antiparallel to the applied magnetic field, from a third magnetic nanoparticle attached to the biological material that is attached to the third receptor,
wherein the fourth magnetic-field sensing element receives a magnetic field, antiparallel to the applied magnetic field, from a fourth magnetic nanoparticle, the fourth magnetic nanoparticle being attached to the biological material that is attached to the fourth receptor, and
wherein a deferential output of a bridge comprising the first, the second, the third and the fourth magnetic-field sensing elements is used to detect at least one of the first magnetic nanoparticle, the second magnetic nanoparticle, the third magnetic nanoparticle and the fourth magnetic nanoparticle by reducing an effect of the applied magnetic field.

26. The magnetic-field biosensor of claim 23, wherein the first and the second magnetic-field sensing elements are each a giant magnetoresistance element (GMR).

27. The magnetic-field biosensor of claim 23, wherein the first and the second magnetic-field sensing elements are each serpentine-shaped.

28. The magnetic-field biosensor of claim 23, wherein the first and the second magnetic-field sensing elements are each a tunneling magnetoresistance (TMR) element.

29. The magnetic-field biosensor of claim 23, wherein the biological material is a piece of deoxyribonucleic acid (DNA), a piece of ribonucleic acid (RNA), a protein, a virus, a biological cell and/or a biological molecule.

30. The magnetic-field biosensor of claim 23, wherein the first and the second magnetic nanoparticles form a magnetic flux concentrator.
